(19) United States
(12) Patent Application Publication
(10) Pub. No.: US 2026/0335577 A1
(43) Pub. Date: Oct. 29, 2026

(54) SYSTEMS AND METHODS FOR DETECTING A LEAK IN A RESPIRATORY THERAPY SYSTEM

(71) Applicant: ResMed Digital Health Inc., San Diego, CA (US)

(72) Inventors: Redmond Shouldice, Dublin (IE); Roxana Tiron, Dublin (IE); Stephen McMahon, Dublin (IE)

(21) Appl. No.: 18/998,929

(22) PCT Filed: Jul. 26, 2023
(86) PCT No.: PCT/IB2023/057602

§ 371 Date: Jan. 27, 2025

Related U.S. Application Data
(60) Provisional application No. 63/393,198, filed on Jul. 28, 2022.

(51) Int. Cl. A61M 16/00

(52) U.S. Cl. CPC A61M 16/024; A61M 16/0003; A61M 2016/0027; A61M 2016/0033; A61M 2205/15; A61M 2205/3334; A61M 2205/3375; A61M 2205/3584; A61M 2205/3592; A61M 2205/502; A61M 2205/6072; A61M 2230/62; A61M 2230/63

(57) ABSTRACT

A method for characterizing a leak in a respiratory therapy system comprises identifying a specific model of a user interface and a conduit of the respiratory therapy system. The method further comprises selecting one of a plurality of predefined pressure versus flow curves based on the identified user interface and conduit. The predefined pressure versus flow curve is associated with airflow characteristics of the respiratory therapy system when the identified user interface is coupled to the identified conduit. The method further comprises receiving pressure data and flow data associated with air flowing in the respiratory therapy system. The method further comprises comparing the pressure data and the flow data to the predefined pressure versus flow curve. The method further includes characterizing, based at least in part on the comparing, a leak in the respiratory therapy system that occurred during the use of the respiratory therapy system.

### CROSS-REFERENCE TO RELATED APPLICATIONS

This application claims the benefit of, and priority to, U.S. Provisional Patent Application No. 63/393,198 filed on Jul. 28, 2022, which is hereby incorporated by reference herein in its entirety.

### TECHNICAL FIELD

The present disclosure relates generally to systems and methods for detecting leaks, and more particularly, to systems and methods for detecting leaks in a respiratory therapy system based at least in part on data associated with air flowing through the respiratory therapy system during use.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. During use of the respiratory therapy system, small amounts of air can leak from the respiratory therapy system, reducing the therapy efficacy of the pressurized air and/or causing discomfort to the user which may in turn affect the user's compliance with prescribed therapy. It is often difficult to detect and mitigate such leaks, in particular leaks that are not immediately perceptible to the user but may become perceptible and uncomfortable over a period of time. Some leaks may change when the user changes position of their head and/or whole body (such as rolling from supine to the user's left side or right side). Current leak detections systems are based on generic pressure and flow curves associated with user interface types (e.g., full face masks, nasal masks, nasal pillow masks, etc.), and a generic pressure and flow curve may be used in respect of multiple mask models despite differences in characteristics, such as impedance, associated with different mask models. Often, the generic pressure and flow curve does not accurately correspond to the actual pressure and flow curve of a particular user interface, or user interface and conduit combination. As such, deviation of measured pressure and flow data points for a given user interface from the generic pressure and flow curve, which might otherwise indicate leak, cannot be relied upon to accurately and reliably detect leak. Thus, new systems and methods are needed for characterizing leaks, which can include detecting leaks. The present disclosure is directed to solving these and other problems.

### SUMMARY

According to some implementations of the present disclosure, a method for characterizing a leak in a respiratory therapy system comprises identifying a specific model of a user interface of the respiratory therapy system. In some implementations, the method also includes identifying a specific model of a conduit coupling the user interface to a respiratory therapy device of the respiratory therapy system. The method also includes, based on the identified specific model of the user interface, selecting a first one of a plurality of predefined pressure versus flow curves. The first one of the plurality of predefined pressure versus flow curves is associated with airflow characteristics of the respiratory therapy system when the identified specific model of the user interface is used with the respiratory therapy system. In some implementations, the selection of the first one of the plurality of predefined pressure versus flow curves is also based on the identified specific model of the conduit. In these implementations, the first one of the plurality of predefined pressure versus flow curves is associated with airflow characteristics of the respiratory therapy system when the identified specific model of the user interface is coupled to the identified specific model of the conduit. The method also includes receiving pressure data and flow data associated with air flowing in the respiratory therapy system during use of the respiratory therapy system by a user. The method also includes comparing the pressure data and the flow data to the first one of the plurality of predefined pressure versus flow curves. The method also includes characterizing, based at least in part on the comparing, a leak in the respiratory therapy system that occurred during the use of the respiratory therapy system by the user.

According to some implementations of the present disclosure, a system for characterizing a leak in a respiratory therapy system comprises an electronic interface, a control system, and a memory. The electronic interface is configured to receive data associated with a sleep session of the individual. The memory stores machine-readable instructions. The control system includes one or more processors configured to execute the machine-readable instructions to execute a method. The method includes identifying a specific model of a user interface of the respiratory therapy system. In some implementations, the method also includes identifying a specific model of a conduit coupling the user interface to a respiratory therapy device of the respiratory therapy system. The method also includes, based on the identified specific model of the user interface, selecting a first one of a plurality of predefined pressure versus flow curves. The first one of the plurality of predefined pressure versus flow curves is associated with airflow characteristics of the respiratory therapy system when the identified specific model of the user interface is used with the respiratory therapy system. In some implementations, the selection of the first one of the plurality of predefined pressure versus flow curves is also based on the identified specific model of the conduit. In these implementations, the first one of the plurality of predefined pressure versus flow curves is associated with airflow characteristics of the respiratory therapy system when the identified specific model of the user interface is coupled to the identified specific model of the conduit. The method also includes receiving pressure data and flow data associated with air flowing in the respiratory therapy system during use of the respiratory therapy system by a user. The method also includes comparing the pressure data and the flow data to the first one of the plurality of predefined pressure versus flow curves. The method also includes characterizing, based at least in part on the comparing, a leak in the respiratory therapy system that occurred during the use of the respiratory therapy system by the user.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5A illustrates flow data associated with a user of a respiratory therapy system, according to some implementations of the present disclosure;

FIG. 5B illustrates pressure data associated with a user of a continuous positive airway pressure system, according to some implementations of the present disclosure;

FIG. 5C illustrates pressure data associated with a user of a respiratory therapy system with an expiratory pressure relief module, according to some implementations of the present disclosure;

FIG. 6A illustrates plotted Cartesian coordinates representing the device pressure and the total flow rate expressed as liters per minute, according to some implementations of the present disclosure;

FIG. 6B illustrates a fitted characteristic curve over the plotted Cartesian coordinates of FIG. 5A, according to some implementations of the present disclosure;

FIG. 7 is a flow diagram for a method for characterizing a leak in a respiratory therapy system, according to some implementations of the present disclosure;

FIG. 8A is a plot comparing pressure versus flow data to a predetermined pressure versus flow curve in the absence of a leak in a respiratory therapy system, according to some implementations of the present disclosure; and

FIG. 8B is a plot comparing pressure versus flow data to the predetermined pressure versus flow curve of FIG. 8B in the presence of a leak in a respiratory therapy system, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale and are provided merely to illustrate the instant disclosure. Several aspects of the disclosure are described below with reference to example applications for illustration.

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate, and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea. As will be understood, a sleep session as described herein can alternatively be referred to as a therapy session, during which an individual may receive respiratory therapy, or can comprise or consist of a therapy session.

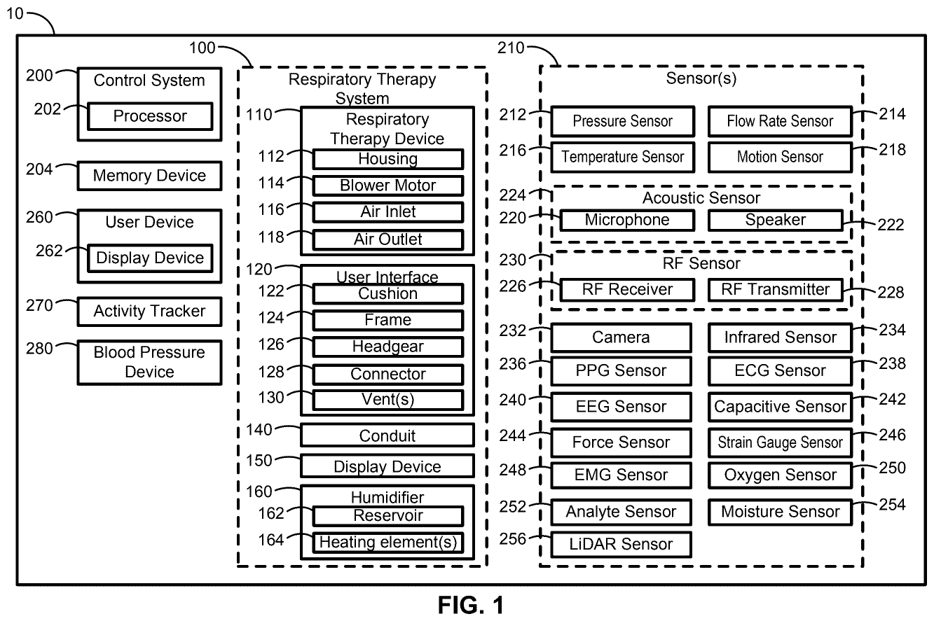

Referring to FIG. 1, a system 10, according to some implementations of the present disclosure, is illustrated. The system 10 can include a respiratory therapy system 100, a control system 200, a memory device 204, and one or more sensors 210. The system 10 may additionally or alternatively include a user device 260, an activity tracker 270, and a blood pressure device 280. The system 10 can be used to detect one or more leaks in the respiratory therapy system 100.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

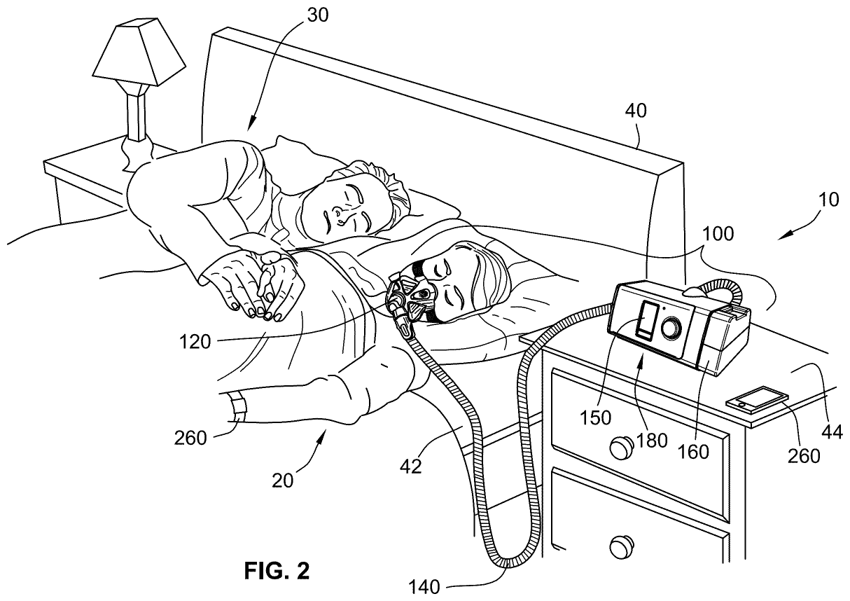

As shown in FIG. 2, the respiratory therapy system 100 can be used to treat a user 20. In this example, the user 20 of the respiratory therapy system 100 and a bed partner 30 are in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

Referring back to FIG. 1, the respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about-20 L/min and about 150 L/min, while maintaining a positive pressure (relative to the ambient pressure).

The respiratory therapy device 110 includes a housing 112, a blower motor 114, an air inlet 116, and an air outlet 118. The blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). The housing 112 can also include a vent to allow air to pass through the housing 112 to the air inlet 116. As described below, the conduit 140 is coupled to the air outlet 118 of the respiratory therapy device 110.

The user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The one or more vents 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full-face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

The conduit 140 includes a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, air flows from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid-state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct). Thus, while the control system 200 and the memory device 204 are shown as independent components in the block diagram of FIG. 1, they may be components of some other component of the system 10, such as the user device 260, the respiratory therapy device 110, etc.

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

The one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared (IR) sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a Light Detection and Ranging (LiDAR) sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the IR sensor 234, the PPG sensor 236, the ECG sensor 238, the EEG sensor 240, the capacitive sensor 242, the force sensor 244, the strain gauge sensor 246, the EMG sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleep stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20, a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 can comprise an acoustic sensor (such as the acoustic sensor 224 discussed herein) and/or an RF sensor (such as the RF sensor 230 discussed herein), which can generate motion data as further discussed herein. In such implementations, the motion sensor 218, the acoustic sensor, and/or the RF sensor can be disposed in a portable device, such as the user device 260 or the portable device 550 discussed herein. Further, while FIG. 1 and FIG. 2 show the respiratory therapy device 110 as including its own display device 150, in some implementations the respiratory therapy device 110 may not include its own display device, as is discussed herein. In some implementations, the motion sensor 218 alternatively or additionally generates one or more signals representing bodily movement of the user, from which may be obtained a signal representing a sleep state of the user, for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. The microphone 220 can be coupled to or integrated in a wearable device, such as a smartwatch, smart glasses, earphones or earbuds, or other head-wearable devices. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones.

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260, and/or can be coupled to or integrated in a wearable device, such as a smartwatch, smart glasses, earphones or ear buds, or other head-wearable devices.

The microphone 220 and the speaker 222 can be used as separate devices. In some implementations, the microphone 220 and the speaker 222 can be combined into an acoustic sensor 224 (e.g., a sonar sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30. Based at least in part on the data from the microphone 220 and/or the speaker 222, the control system 200 can determine a location of the user 20 and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g., a radar sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user, to determine air flow of the mouth and/or nose of the user, to determine a time when the user enters the bed, and to determine a time when the user exits the bed. In some implementations, the camera 232 includes a wide-angle lens or a fisheye lens.

The IR sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120, in the associated headgear (e.g., straps, etc.), in a head band or other head-worn sensor device, etc.

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 252 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 252 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The LiDAR sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 10, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 214 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, a microphone, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be non-physiological parameters. Other types of physiological and non-physiological parameters can also be determined, either from the data from the one or more sensors 210, or from other types of data.

The user device 260 includes a display device 262. The user device 260 can be, for example, a mobile device such as a smartphone, a tablet computer, a gaming console, a smartwatch, a laptop computer, or the like. In some implementations, the user device 260 is a portable device, such as a smartphone, a tablet computer, a smartwatch, a laptop computer, etc. Alternatively, the user device 260 can be an external sensing system, a television (e.g., a smart television), or another smart home device (e.g., a smart speaker(s) such as Google Home, Amazon Echo, Amazon Alexa, etc.). In some implementations, the user device is a wearable device (e.g., a smartwatch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 10 also includes the activity tracker 270. The activity tracker 270 is generally used to aid in generating physiological data associated with the user. The activity tracker 270 can include one or more of the sensors 210 described herein, such as, for example, the motion sensor 218 (e.g., one or more accelerometers and/or gyroscopes), the PPG sensor 236, and/or the ECG sensor 238. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 10 also includes the blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp, chirp, triangle, sinewave, etc.), other schemes such as PSK, FSK etc., pulsed continuous wave, and/or spread in ultra-wideband ranges (which may include spreading, PRN codes or impulse systems).

While the control system 200 and the memory device 204 are described and shown in FIG. 1 as being a separate and distinct component of the system 10, in some implementations, the control system 200 and/or the memory device 204 are integrated in the user device 260 and/or the respiratory therapy device 110. Thus, the control system 200 and/or the memory device 204 can be disposed within the housing 112 of the respiratory therapy device 110. Alternatively, in some implementations, the control system 200 or a portion thereof (e.g., the processor 202) can be located in a cloud (e.g., integrated in a server, integrated in an Internet of Things (IOT) device, connected to the cloud, be subject to edge cloud processing, etc.), located in one or more servers (e.g., remote servers, local servers, etc., or any combination thereof.

While system 10 is shown as including all the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

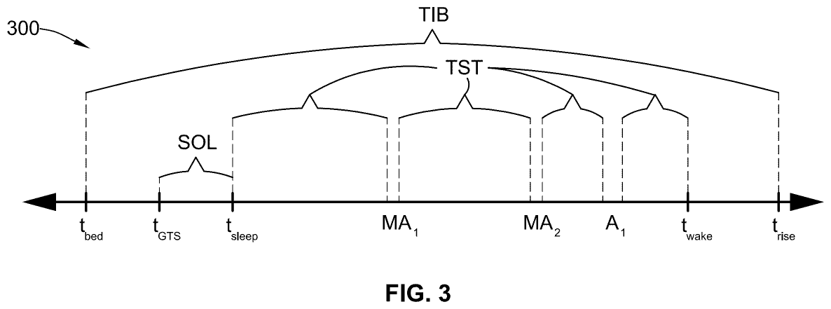

Referring now to FIG. 3, as used herein, a sleep session can be defined multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user has laid or sat down in the bed (or another area or object on which they intend to sleep) and has turned on the respiratory therapy device 110 and donned the user interface 120. The sleep session can thus include time periods (i) when the user is using the respiratory therapy system 100, but before the user attempts to fall asleep (for example when the user lays in the bed reading a book); (ii) when the user begins trying to fall asleep but is still awake; (iii) when the user is in a light sleep (also referred to as stage 1 and stage 2 of non-rapid eye movement (NREM) sleep); (iv) when the user is in a deep sleep (also referred to as slow-wave sleep, SWS, or stage 3 of NREM sleep); (v) when the user is in rapid eye movement (REM) sleep; (vi) when the user is periodically awake between light sleep, deep sleep, or REM sleep; or (vii) when the user wakes up and does not fall back asleep. The sleep session may also be referred to as a therapy session, or may comprise a therapy session, which can be understood to be the period of time within the sleep session during which the individual is engaged in respiratory therapy (e.g., the use of a respiratory therapy system).

The sleep session is generally defined as ending once the user removes the user interface 120, turns off the respiratory therapy device 110, and gets out of bed. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user, ending when the respiratory therapy device 110 stops supplying the pressurized air to the airway of the user, and including some or all the time points in between, when the user is asleep or awake.

FIG. 3 illustrates an exemplary timeline 300 for a sleep session. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 40 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}), or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, as shown in the timeline 300, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

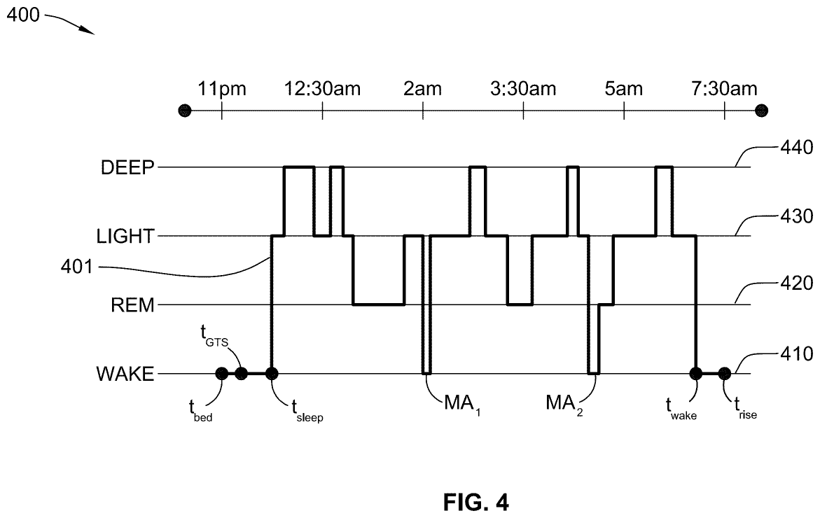

Referring to FIG. 4, an exemplary hypnogram 400 corresponding to the timeline 300 of FIG. 3, according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based at least in part on physiological data associated with the user (e.g., generated by one or more of the sensors 210 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 204.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 218, the microphone 220, the camera 232, or any combination thereof. The go-to-sleep time can be determined based at least in part on, for example, data from the motion sensor 218 (e.g., data indicative of no movement by the user), data from the camera 232 (e.g., data indicative of no movement by the user and/or that the user has turned off the lights), data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

As noted herein, the system 10 can include a flow rate sensor (e.g., the flow rate sensor 214 of FIG. 1) and/or a pressure sensor (e.g., the pressure sensor 212 of FIG. 1). The flow rate sensor 134 can be used to generate flow data associated with the user 20 (FIG. 2) of the respiratory therapy device 110 during the sleep session. In some implementations, the flow rate sensor 134 is configured to measure a vent flow (e.g., intentional “leak”), an unintentional leak (e.g., mouth leak (e.g., a leak from the mouth when using a nasal mask or a nasal pillow mask) and/or mask leak), a patient flow (e.g., air into and/or out of lungs), or a combination thereof. In some implementations, the flow data can be analyzed to determine cardiogenic oscillations of the user.

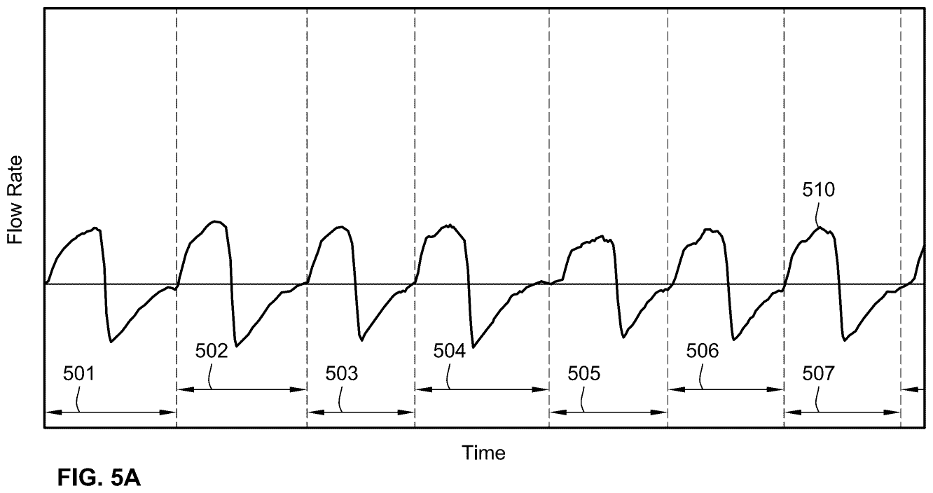

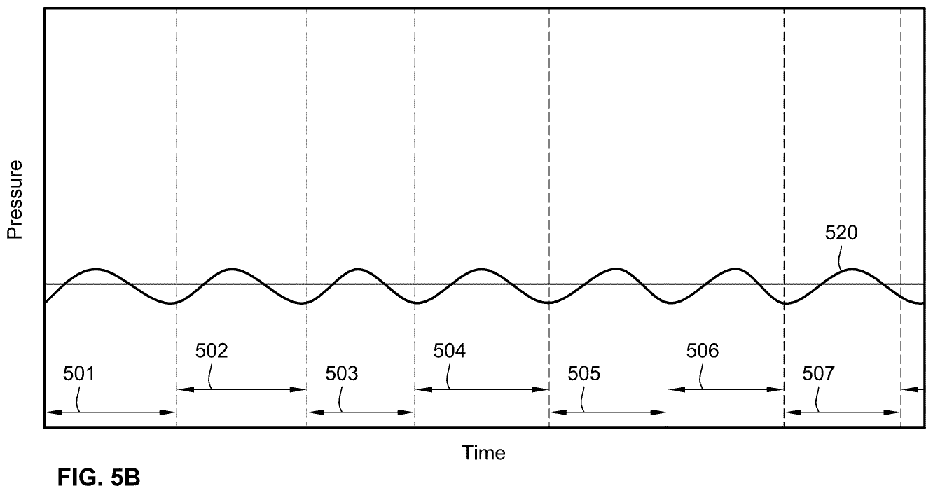

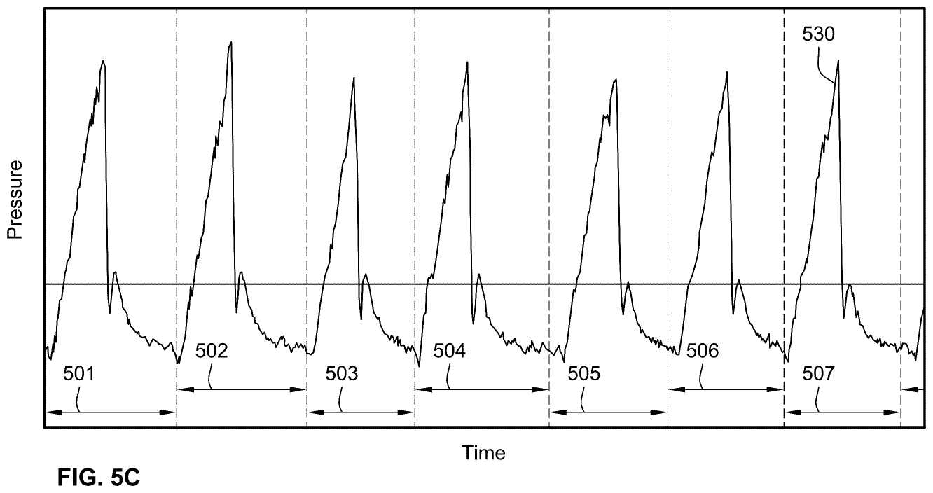

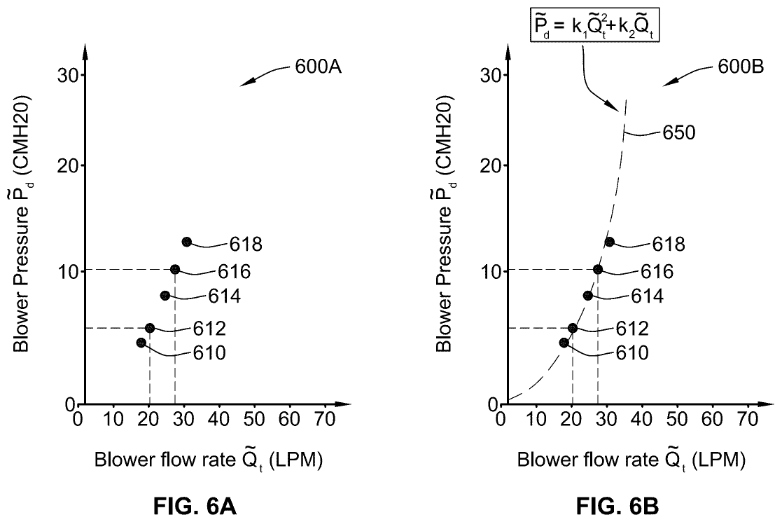

In some implementations, the flow rate sensor and/or a pressure sensor are configured to generate flow data over a period of therapy time. For example, FIG. 5A illustrates a portion of such flow data associated with a user (e.g., the user 20 of FIG. 2) of a respiratory therapy system (e.g., the respiratory therapy system 100 of FIG. 1), according to some implementations of the present disclosure. As shown in FIG. 6A, a plurality of flow rate values measured over about seven full breathing cycles (501-507) is plotted as a continuous curve 510.

In some implementations, the pressure sensor is configured to generate pressure data over a period of therapy time. For example, FIG. 5B illustrates pressure data associated with a user of a CPAP system, according to some implementations of the present disclosure. The pressure data shown in FIG. 5B was generated over the same period of therapy time as that of FIG. 5A. As shown in FIG. 5B, a plurality of pressure values measured over about seven full breathing cycles (501-507) is plotted as a continuous curve 520. Because a CPAP system is used, the continuous pressure curve of FIG. 5B exhibits a generally sinusoidal pattern with a relatively small amplitude, because the CPAP system attempts to maintain the constant predetermined air pressure for the system during the seven full breathing cycles.

Referring to FIG. 5C, pressure data associated with a user of a respiratory therapy system with an expiratory pressure relief (EPR) module is illustrated, according to some implementations of the present disclosure. The pressure data shown in FIG. 5C was generated over the same period of therapy time as that of FIG. 5A. As shown in FIG. 5C, a plurality of pressure values measured over about seven full breathing cycles (501-507) is plotted as a continuous curve 530. The continuous curve 530 of FIG. 5C is different from the continuous curve 520 of FIG. 5B, because the EPR (Expository Pressure Relief) module (used for the pressure data in FIG. 5C) can have different settings for an EPR level, which is associated with a difference between a pressure level during inspiration and a reduced pressure level during expiration.

Referring now to FIGS. 6A and 6B, flow data and/or pressure data can be used to generate pressure versus flow curves (also referred to as P-Q curves) that can be used for a variety of different purposes. As discussed herein, in some implementations, a pathway is formed by a respiratory therapy device (e.g., the respiratory therapy device 110), a mask (e.g., the user interface 120), and a conduit (e.g., the conduit 140). The conduit creates a first impedance Z1 (humidifier tub, tube, and mask impedance), which in turn causes a pressure drop ΔP that is a function of the total flow rate Qt. The user interface (e.g., mask) pressure Pm is the device pressure Pd (also referred to as the blower pressure) less the pressure drop ΔP through the conduit.

Pm=Pd−ΔP Eq. 1

where ΔP is the pressure drop, characteristic of the conduit. The pressure drop ΔP is a function of the total flow rate Qt (also referred to as the blower flow rate):

ΔP=Z1·Qt Eq. 2

In some implementations, the vent of the mask creates a second impedance Z2 (vent impedance). The mask interface pressure Pm is directly related to the vent flow rate Qu via the vent impedance characteristic Z2:

Pm=Z2·Qv Eq. 3

Similarly, the vent flow rate Qu is directly related to the mask interface pressure Pm via the vent admittance characteristic Y2:

$$Y 2 = \frac{1}{Z 2} \qquad Eq . 4$$

$$Qv = Y 2 \cdot Pm \qquad Eq . 5$$

Combining equation (1) with equation (5), the vent flow rate Qu may be determined as:

Qv=Y2·(Pd−ΔP) Eq. 6

An unintentional leak, which is unknown and unpredictably variable, creates a third impedance Z3 (leak impedance). Additionally, in some implementations, the fourth impedance (airway resistance and lung compliance) includes (i) the patient airway resistance Z4, (ii) the patient lung compliance Clung, and/or (iii) the variable pressure source Plung, each of which represents characteristics of the user's respiratory circuit. Thus, the total flow rate Qt (also referred to as the blower flow rate) is equal to the sum of the vent flow rate Qu, the leak flow rate Qleak, and the respiratory flow rate Qr:

Qt=Qv+Qleak+Qr Eq. 7

In some implementations, the respiratory flow rate Qr averages to zero over a plurality of respiratory cycles (e.g., breathing cycles), because the average respiratory flow rate into or out of the lungs must be zero. The average respiratory flow rate generally removes the influence of the user's breathing on the flow rate. In other implementations, the user's breathing can be removed from the flow rate in other manners Taking tilde (~) to indicate the value with the user's breathing removed:

Õr=0 Eq. 8

As such, the leak flow rate with the user's breathing removed may be approximated as:

Õleak=Õt−Õv Eq. 9

In implementations where averaging is used, the process of averaging may be implemented by low-pass filtering with a time constant long enough to contain the plurality of respiratory cycles. The time constant can be of any suitable duration, such as five seconds, ten seconds, thirty seconds, one minute, etc. However, other time intervals are also contemplated.

Combining equations (1), (2), and (3), the device pressure absent the user's breathing \tilde{P}d (also referred to as the blower pressure absent the user's breathing) may be written as:

\tilde{P}d=Z1·\tilde{Q}t+Z2·\tilde{Q}v Eq. 10

In the absence of any leak flow (e.g., \tilde{Q}leak=0), the total flow rate absent the user's breathing \tilde{Q}t is equal to the vent flow rate absent the user's breathing \tilde{Q}v. Equation (10) can then be written to reflect the relationship between the total flow rate \tilde{Q}t and the device pressure \tilde{P}d that characterizes the respiratory therapy air circuit:

\tilde{P}d=Z1·\tilde{Q}t+Z2·\tilde{Q}t Eq. 11

\tilde{P}d=(Z1+Z2)·\tilde{Q}t Eq. 12

\tilde{P}d=(Z1+Z2)·\tilde{Q}v Eq. 13

The relationship is determined by the vent impedance characteristic Z2 and the conduit pressure drop impedance characteristic Z1. In the presence of leak, \tilde{Q}t>\tilde{Q}v. In the absence of leak, \tilde{Q}t=\tilde{Q}v. Thus, in an idealized situation where the respiratory therapy system includes no leaks, all the pressurized air that flows into and out of the user's mouth will then flow through one or more vents of the respiratory therapy system. However, if there any leaks in the respiratory therapy system, some of the air that would otherwise flow out of the respiratory therapy system through one or more vents of the respiratory therapy system will instead flow out of the respiratory therapy system at the location of the leak(s).

Referring now to FIG. 6A, a scatter plot 600A of total flow rate \tilde{Q}t (in liters per minute) versus device pressure \tilde{P}d (in cmH₂O) is depicted. FIG. 6A illustrates plotted Cartesian coordinates representing device pressure and total flow rate, expressed as liters per minute (“LPM,” which is equivalent to 60 times L/s). Each Cartesian coordinate includes an X value and a Y value. As shown, five Cartesian coordinates 610, 612, 614, 616, and 618 are plotted in the scatter plot 600A over a period of therapy. Each Cartesian coordinate may also be expressed as (\tilde{Q}t, \tilde{P}d). The device (blower) pressure \tilde{P}d and the total (blower) flow rate \tilde{Q}t can represent filtered pressure and flow rate values in the sense that the influence of the user's breathing on the pressure and flow rate data has been filtered out, such as achieved by averaging or by sampling (or selecting) the pressure and flow rate values at breathing cycle transitions (e.g., points of transition between inspiration and expiration and/or between expiration and inspiration) as described herein.

For example, the first Cartesian coordinate 612 has a first X value that is about 20 liters per minute, and a first Y value that is about 6 cmH₂O. As such, the first Cartesian coordinate 612 can be expressed as (20, 6). The first X value can be estimated and/or calculated based at least on a first plurality of flow rate values generated over a first time period. In some implementations, the first X value is an average flow rate value of the first plurality of flow rate values generated over the first time period.

In some implementations, the first time period is a predetermined time interval, such as five seconds, ten seconds, 30 seconds, one minute, two minutes etc. In some implementations, the first time period includes one or more full breathing cycles, such as one breathing cycle, two breathing cycles, five breathing cycles, ten breathing cycles, etc. Therefore, in some implementations, the first X value is the average flow rate value of the first plurality of flow rate values generated over one or more breathing cycles, such as seven breathing cycles (FIG. 6A).

Similarly, the first Y value can be estimated and/or calculated based at least on a first plurality of pressure values generated over the first time period. Each of the first plurality of pressure values corresponds with a respective one of the first plurality of flow rate values. For example, in some implementations, each of the first plurality of flow rate values has a corresponding time stamp (e.g., FIG. 5A). Using the corresponding time stamp, the respective one of the first plurality of pressure values can be identified (e.g., FIG. 5B or FIG. 5C). Therefore, in some implementations, the first Y value is the average pressure value of the first plurality of pressure values generated over one or more breathing cycles, such as seven breathing cycles (e.g., FIG. 5B or FIG. 5C).

The second Cartesian coordinate 616 has a second X value that is about 28 liters per minute, and a second Y value that is about 10 cmH₂O. As such, the second Cartesian coordinate 616 can be expressed as (28, 10). The second X value and the second Y value of the second Cartesian coordinate 616 can be estimated and/or calculated the same way as, or similar to, the first X value and the first Y value of first Cartesian coordinate 612.

Referring to FIG. 6B, based at least in part on the first Cartesian coordinate 612 and the second Cartesian coordinate 616, a pressure versus flow rate curve 650 can be fitted in the plot 600B. For example, in some implementations, the pressure versus flow rate curve 650 may be approximated using a polynomial equation, such as a quadratic equation:

$${\tilde{P}}_{d} = {k}_{1} \cdot {\tilde{Q}}_{v}^{2} + {k}_{2} \cdot {\tilde{Q}}_{v} \qquad Eq . 14$$

The parameters of the pressure versus flow rate curve, in this quadratic equation, two non-zero constants (or coefficients), k₁ and k₂, characterize the series concatenation of the vent impedance characteristic Z1 and the air circuit pressure drop impedance characteristic Z2. In some implementations, the polynomial equation defines an intentional leak of the system (e.g., vent flow of the system) by providing a corresponding flow rate of intentional leak for a given pressure.

If at least two Cartesian coordinates are known, the non-zero constants k₁ and k₂ can be solved. For example, using the first Cartesian coordinate 812 (20, 6) and the second Cartesian coordinate 816 (28, 10), equation (14) can be solved and re-written as approximately:

$${\tilde{P}}_{d} = \frac{1}{140} {\tilde{Q}}_{v}^{2} + \frac{11}{70} {\tilde{Q}}_{v} \qquad Eq . 15$$

In other words, the non-zero constant k₁ is 1 (about 0.00714), and the non-zero constant k₂ is 11/70 (about 0.157) for the quadratic curve going through the first Cartesian coordinate 812 and the second Cartesian coordinate 816. Therefore, in some implementations, the pressure versus flow rate curve 850 can be estimated and/or defined as equation (15).

In some implementations, the non-zero constants depend on (i) the unit for the pressure values, (ii) the unit for the flow rate values, (iii) the vent for the respiratory therapy system, (iv) the mask for the respiratory therapy system, (v) the humidifier tub for the respiratory therapy system, (vi) the conduit for the respiratory therapy system, (vii) computation scaling factors, or (viii) any combination thereof. The non-zero constants can also vary with different types of masks, different models of masks, different manufacturers of masks, and/or different batches of masks. For example, with the pressure values measured in cmH₂O and the flow rate values measured in L/min, the non-zero constants k₁ and k₂ can be about 1/154 and about 1/7 respectively, for a particular vent of a respiratory therapy system.

In some implementations, the polynomial equation may have more than two non-zero constants, such as three non-zero constants, four non-zero constants, five non-zero constants, etc. For example, the polynomial equation may be expressed as:

$${\tilde{P}}_{d} = {k}_{1} \cdot {\tilde{Q}}_{v}^{2} + {k}_{2} \cdot {\tilde{Q}}_{v} + {k}_{3} \qquad Eq . 16$$

In some implementations, the polynomial equation may involve a power of three, four, five, etc. For example, the polynomial equation may be expressed as:

$${\tilde{P}}_{d} = {k}_{1} \cdot {\tilde{Q}}_{v}^{3} + {k}_{2} \cdot {\tilde{Q}}_{v}^{2} + {k}_{3} \cdot {\tilde{Q}}_{v} + {k}_{4} \qquad Eq . 17$$

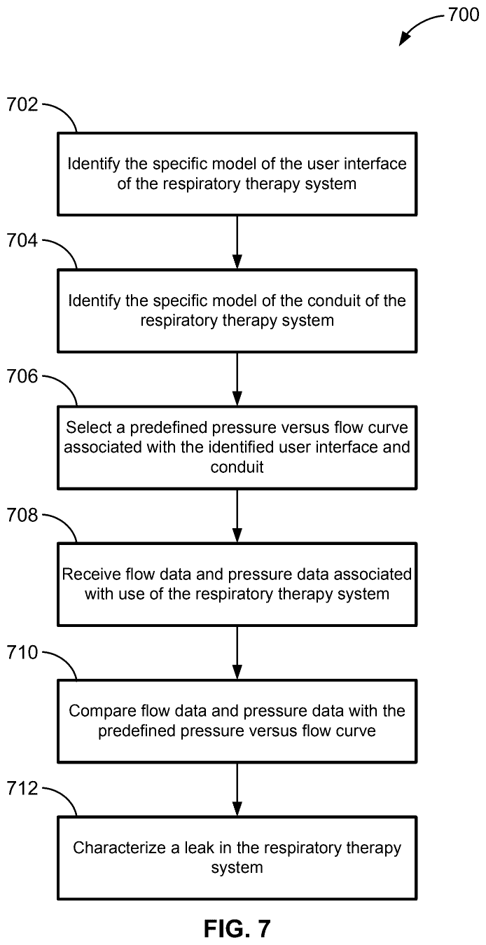

FIG. 7 illustrates a method 700 for characterizing one or more leaks in a respiratory therapy system (such as the respiratory therapy system 100). As noted above, the respiratory therapy system can be designed such that there is a flow of air out of a vent somewhere in the respiratory therapy system (e.g., a user interface of the respiratory therapy system (such as the user interface 120), a conduit of the respiratory therapy system (such as the conduit 140), a respiratory therapy device of the respiratory therapy system (such as the respiratory therapy device 110), etc.). This flow can be referred to as an intentional leak and is expected as part of the normal operation of the respiratory therapy system. However, unintentional leaks can also occur somewhere in the respiratory therapy system. These unintentional leaks can be difficult to detect and can lead to the respiratory therapy system offering less effective respiratory therapy to the user and/or cause discomfort to the user even if not immediately perceptible by the user. For example, the unintentional leak may only change when the user is in a state of sleep (e.g., the unintentional leak is low when awake, but increases when asleep as the user changes position, and/or with changes in the therapy pressure applied, etc.). Method 700 includes analyzing pressure and flow data of the respiratory therapy system to compare the actual performance of the respiratory therapy system to the ideal performance of the respiratory therapy system (e.g., without any unintentional leaks), in order to characterize any unintentional leaks in the respiratory therapy system. Generally, a control system (such as the control system 200 of the system 10) is configured to carry out the various steps of method 700. A memory device (such as the memory device 204 of the system 10) can be used to store any type of data utilized in the steps of method 700 (or other methods). As used herein, the term “leak” will refer to an unintentional leak, unless otherwise noted.

Step 702 of the method 700 includes identifying the specific model of the user interface that is being used with the respiratory therapy system. Similarly, step 704 of the method 700 includes identifying the specific model of the conduit that is used to couple the user interface to the respiratory therapy device of the respiratory therapy system. Generally, a variety of different types of user interfaces and conduits can be used with the respiratory therapy system. The specific type and model of user interface and conduit can affect the expected performance of the respiratory therapy system (e.g., the performance of the respiratory therapy system in the absence of any leaks). The specific type and model of user interface and conduit can have associated impedance characteristics which affect such performance. The specific model of the user interface can be a model that is selected from a cohort of models of user interfaces that the user may use with the respiratory therapy system. The specific model of the user interface has an associated intentional leak characteristic curve, which may be in the form of a pressure versus flow curve. Similarly, the specific model of the conduit can be a model that is selected from a cohort of models of conduits that the user may use with the respiratory therapy system. A combination of the specific model of the user interface and the specific model of the conduit has an associated intentional leak characteristic curve, which may be in the form of a pressure versus flow curve.

The user interface can be differentiated in a number of different ways. For example, the user interface can belong to a specific family of user interfaces. Different user interface families can include a full-face mask, a partial face mask, a nasal mask, nasal pillows, a total-face mask (which may cover, in addition to the user's mouth and nose, some or all of the user's eyes and/or ears). The user interface can additionally or alternatively have various sizes, such as extra-small, small, medium, large, extra-large or any reasonable intermediary size such as small/medium, etc. The user interface can additionally or alternatively belong to a specific style of user interface. The user interface style generally refers to either a face-mounted user interface or a conduit style user interface. A face-mounted style user interface generally refers to a user interface that is generally positioned on the front of the user's face, with the conduit attached to the front of the user interface at the front of the user's face. The face-mounted style user interface may include one or more straps that extend around the user's head to secure the user interface to the user's head. A conduit style user interface, also referred to as a headgear user interface, is a user interface that includes its own conduit that extends around the user's head. The conduit from the respiratory therapy device is coupled to the conduit of the conduit style user interface at the top of the user's head. The conduit of the conduit style user interface then extends to the front of the user's face and is positioned in front of the user's mouth and/or nose, to provide air to the user's airway. In some implementations, the conduit style user interface has a single conduit that extends on one side of the user's face. In other implementations, the conduit style user interface has two conduits that extend on either side of the user's face. User interfaces can also be differentiated by manufacturer, number of vents, size of vents, materials used to construct the user interface, the presence of electrical contacts that can be used to electrically connect the user interface to other components (such as the respiratory therapy device), and any other number of features.

In specific examples, the user interface may be selected from the following: AcuCare™ F1-0 non-vented (NV) full face mask, AcuCare™ F1-1 non-vented (NV) full face mask with AAV, AcuCare™ F1-4 vented full face mask, AcuCare™ high flow nasal cannula (HFNC), AirFit™ F10, AirFit™ F20, AirFit™ F30, AirFit™ F30i, AirFit™ masks for AirMini™, AirFit™ N10, AirFit™ N20, AirFit™ N30, AirFit™ N30i, AirFit™ P10, AirFit™ P30i, AirTouch™ F20, AirTouch™ N20, Mirage Activa™, Mirage Activa™ LT, Mirage™ FX, Mirage Kidsta™, Mirage Liberty™, Mirage Micro™, Mirage Micro™ for Kids, Mirage Quattro™, Mirage SoftGel™, Mirage Swift™ II, Mirage Vista™, Pixi™, Quattro™ Air, Quattro™ Air NV, Quattro™ FX, Quattro™ FX NV, ResMed™ full face hospital mask, ResMed™ full face hospital NV (non-vented) mask, ResMed™ hospital nasal mask, Swift™ FX, Swift™ FX Bella, Swift™ FX Nano, Swift™ LT, Ultra Mirage™, Ultra Mirage™ II, Ultra Mirage™ NV (non-vented) full face mask, Ultra Mirage™ NV (non-vented) nasal mask, for example.

The conduit being used with the respiratory therapy system can also be differentiated in a number of different ways. For example, the conduit may be of a specific length and/or diameter. The length and/or diameter can be defined by a numerical measurement (e.g., number of inches, number of feet, number of centimeters, number of meters, etc.). The length and/or diameter can also be defined by a descriptive category (e.g., extra-small, small, medium, large, extra-large or any reasonable intermediary size such as small/medium, etc.). Conduits can also be differentiated by manufacturer, number of vents, size of vents, materials used to construct the conduit, the presence of heating components used to heat the pressurized air, the presence of electrical contacts to electrically connect the conduit to other components (such as the user interface and/or the respiratory therapy device), and any other number of features. In specific examples, the conduit may be selected from the following: ResMed™ ClimateLine™ ResMed™ Slimline™, for example.

In some cases, identifying the specific model of user interface and conduit can refer to determining and/or identifying any features or other factors of the user interface and conduit that can affect the flow, the pressure, and/or the impedance in respect of the pressurized air flowing through the respiratory therapy system when user that combination of user interface and conduit.

The specific model of user interface and conduit can be identified in any number of suitable ways. For example, in some implementations, the user interface and/or conduit can include some type of identifying marker, such as a radio-frequency identification (RFID) tag, a Bluetooth Low Energy (BLE) tag, a barcode, a QR code, etc. The identifying marker can be read, scanned, or otherwise analyzed to determine the model of the user interface and/or conduit. The identifying marker could be positioned inside of the user interface and/or conduit or outside of the user interface and/or conduit (e.g., an RFID tag or a BLE tag). The identifying marker could also be printed on the surface of the user interface and/or conduit (e.g., a barcode or a QR code).

In other implementations, the system may analyze an image of the user interface and/or the conduit to identify the models of the user interface and/or conduit. This analysis could be performed using any suitable image recognition algorithm. In further implementations, the system can analyze flow data and/or pressure data generated using the respiratory therapy system with the user's user interface and conduit in order to identify the model of the user interface and/or conduit. In other implementations, the system can generate acoustic data that is associated with one or more vents of a user interface and/or conduit. In still other implementations, the system can generate acoustic data that is associated with one or more acoustic reflections of an acoustic signal propagating within the user interface and/or conduit. The acoustic reflections are generally indicative of, at least in part, features of the user interface and/or conduit. The acoustic data can be analyzed to identify the specific model of the user interface and/or conduit. Additional details related to using flow data, pressure data, and acoustic data to identify the user interface and/or conduit are described in WO 2021/245637, WO 2021/250553, and PCT/IB2022/053332, each of which is hereby incorporated by reference herein in its entirety. In even further implementations, the user can manually input the specific model of the user interface and/or conduit, for example via a user device (such as the user device 260). The user could additionally or alternatively input specific features and/or characteristics of the user interface and/or conduit.

At step 706 of method 700, a predefined pressure versus flow curve is selected from a plurality of predefined pressure versus flow curves. Each of the plurality of predefined pressure versus flow curves is associated with airflow characteristics of a respiratory therapy system with a specific user interface, or a specific combination of user interface and conduit, and generally represents the expected performance of that user interface/conduit combination in the absence of any leaks in the respiratory therapy system. As described herein, the predefined pressure versus flow curve can be described as the intentional leak characteristic curve for a specific user interface or specific combination of user interface and conduit. The plurality of predefined pressure versus flow curves can include at least 3 curves, at least 10 curves, at least 25 curves, at least 50 curves, at least 100 curves, at least 500 curves, etc., wherein each curve corresponds to a specific user interface, or a specific combination of user interface and conduit. In any of these implementations, there will generally be only one predefined pressure versus flow curve that corresponds to the combination of the identified model of the user interface and the identified model of the conduit. However, in some cases, there may be more than one pressure versus flow curve that can correspond to the identified user interface and conduit combination, for example if the identification of the user interface and/or conduit it is more general (e.g., identification of the user interface as a specific model and manufacturer, compared to identification of the user interface as simply a nasal mask). In these implementations, only a single one of these pressure versus flow curves can be used analyze the respiratory therapy system, or multiple pressure versus flow curves can be used to analyze the respiratory therapy system.

At step 708 of the method 700, flow data and pressure data associated with the user of the respiratory therapy system is received. The flow data can include one or more flow rate values associated with the pressurized air flowing through the respiratory therapy system, for example as discussed herein with respect to FIG. 5A. The flow rate values can include flow rate values of air flowing between the respiratory therapy device and the conduit, flow rate values of air flowing between the conduit and the user interface, flow rate values of air flowing into and out of the user's airway (e.g., into and out of the user's mouth and/or nose), flow rate values of air flowing into and out any vents in the user interface or other components of the respiratory therapy system, and other flow rate values. The flow data can be used to generate flow rate versus time curves, such as the flow rate versus time curve illustrated in FIG. 5A. The pressure data can include one or more pressure values associated with the pressurized air flowing through the respiratory therapy system, for example as discussed herein with respect to FIGS. 5B and 5C. The pressure data can be used to generate pressure versus time curves, such as the pressure versus time curves illustrated in FIGS. 5B and 5C.

At step 710 of the method 700, the flow data and pressure data are compared to the selected predefined pressure versus flow curve. At step 712 of the method 700, a leak in the respiratory therapy system is characterized based at least in part on the comparison. In some implementations, the flow data and the pressure data can be plotted on a pressure versus flow graph and compared to the selected pressure versus flow curve. Differences between the data points and the selected pressure versus flow curve (which represents the expected performance of the combination of the user interface and conduit in the absence of any leaks in the respiratory therapy system) can be used to characterize to one or more leaks that may be occurring in the respiratory therapy system.

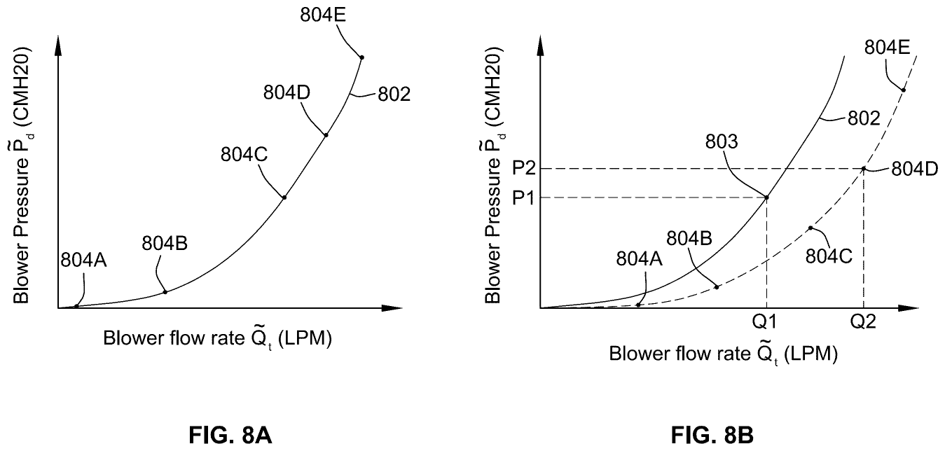

FIGS. 8A and 8B illustrate how the flow data and the pressure data can be compared to the predefined pressure versus flow curve to characterize any leaks in the respiratory therapy system in steps 710 and 712. FIGS. 8A and 8B show pressure versus flow plots with pressure plotted on the vertical axis, and flow rate plotted on the horizontal axis. Each pressure value represents the device pressure, e.g., the pressure of the air generated by a motor of the respiratory therapy device (such as the blower motor 114 of the respiratory therapy device 110) and is the sum of (i) the pressure at the user interface and (ii) the pressure drop across the conduit and the user interface. Each flow rate value represents the flow rate at the motor of the respiratory therapy device and is the sum of (i) the flow rate through the vent(s) of the respiratory therapy system and (ii) the flow rate of an unintentional leaks, after the flow rate due to the user's breathing has been removed.

The pressure versus flow plot includes the selected predefined pressure versus flow curve 802, and a plurality of pressure versus flow data points 804A-804E. Each of the pressure versus flow data points 804A-804E correspond to the device pressure and the flow rate at a given moment in time. Thus, the pressure versus flow data points 804A-804E are generally similar to the Cartesian coordinates 610-618 of FIGS. 6A and 6B and can be obtained in a similar fashion. The pressure versus flow curve 802 shows the device pressure required to achieve a certain flow rate. Because the pressure versus flow curve 802 represents the flow rate in a respiratory therapy system with no unintentional leak (or a negligible amount of unintentional leak), the flow rate represented by the pressure versus flow curve 802 is the flow through the vent(s) of the respiratory therapy system.

In FIG. 8A, the pressure versus flow data points 804A-804E generally match the predefined pressure versus flow curve 802. For each pressure indicated by the data points 804A-804E, the corresponding flow rate generally has the value expected when there are no leaks in the respiratory therapy system. Thus, when the data points 804A-804E generally align with the predefined pressure versus flow curve 802, it can be determined that there is no leak in the respiratory therapy system.

In FIG. 8B, the pressure versus flow data points 804A-804E do not align with the predefined pressure versus flow curve 802, but instead are shifted away from the predefined pressure versus flow curve 802. For example, due to the presence of the leak, the flow rate values will increase. To ensure that the pressure at the user interface remains the same (relative to the pressure at the user interface in the absence of any leak), the respiratory therapy system will compensate for the leak by increasing the pressure at the blower motor of the respiratory therapy device. The measured pressure values will thus also increase. The change in the pressure and flow rate values is shown in FIG. 8B. For a respiratory therapy system with no leak characterized by the predefined pressure versus flow curve 802, a given pressure P1 results in a flow rate Q1, which intersect at point 803 on the pressure versus flow curve 802. However, in a system with a leak characterized by data points 804A-804E, the flow rate values and the pressure values increase. Due to the leak, the flow rate Q1 increases to flow rate Q2, and the respiratory therapy system increases the pressure from P1 to P2 to compensate. Generally, the difference between Q1 and Q2 is the flow rate of the leak, and the difference between P1 and P2 is the increased pressure applied by the respiratory therapy system to compensate for the leak. Thus, by plotting the pressure versus flow data points (such as data points 804A-804E) and comparing them to the predefined pressure versus flow curve (such as the pressure versus flow curve 802), a leak in the respiratory therapy system can be detected due to the difference between the pressure versus flow data points and the predefined pressure versus flow curve.

Generally, the method 700 can be used to detect leaks of various sizes. For example, the detected leak may have a flow rate of between about 0.001 liters per minute and about 5.0 liters per minute, optionally between about 0.001 liters per minute and about 2.0 liters per minute, further optionally between about 0.001 liters per minute and about 1.0 liters per minute. In further examples, the detected leak may have a flow rate of less than or equal to about 5.0 liters per minute, optionally less than or equal to about 2.0 liters per minute, further optionally less than or equal to about 1.0 liter per minute. In such examples, leaks having a flow rate up to or greater than about 5.0 liters per minute can also be detected using the method 700.

Referring back to FIG. 7, method 700 can be implemented in real-time, in a delayed fashion during the sleep session, after the sleep session has been completed, or any combination thereof. For example, if implemented in real-time or in a delayed fashion, method 700 can be used to characterize leaks as they occur (or some time thereafter) during the sleep session. In these implementations, method 700 can further include taking action(s) to mitigate the leak during the sleep session, analyzing additional pressure data and the flow data received during the sleep session to determine additional information related to the leak and other steps. In these implementations, method 700 can be used to conduct a fit test of the user interface before the user has fallen asleep during the sleep session. In such an implementation, after the user has donned the user interface and begun to use the respiratory therapy system, the system can analyze the data to determine if any leaks are occurring. If leaks are occurring, the user can adjust the fit of the user interface to see if the leak has been mitigated. Thus, the user can improve the fit of the mask while awake, so that leaks do not occur when the user is asleep, which could cause discomfort and/or reduce the intended therapy effect of the respiratory therapy system. If implemented after the sleep session has finished, method 700 can further include transmitting recommendations and/or instructions to the user to aid in reducing leaks during subsequent sleep sessions.

In some implementations, characterizing the leak at step 712 can include a variety of different actions. In some implementations, characterizing the leak includes detecting the presence of the leak somewhere in the respiratory therapy system. However, characterizing the leak can additionally or alternatively include estimating the location of the leak within the respiratory therapy system, and/or determining the flow rate of the leak.

Estimating the location of the leak can be achieved in a variety of different ways. For example, in some implementations, acoustic data can be generated that is representative of noise associated with operating of the respiratory therapy system. The acoustic data can be generated using a one or more microphones (such as the microphone 220 of the system 10) located within the respiratory therapy system, or any other suitable sensor or device. The acoustic data can be analyzed to determine an acoustic signature that is associated with the leak. For example, leaks at different locations within the respiratory therapy system may generally have different characteristics, such that the acoustic signature of a leak at a given location (e.g., within the user interface, between the user interface and the conduit, within the conduit, between the conduit and the respiratory therapy device, within the respiratory therapy device, etc.) is distinct from the acoustic signature of a leak at some other location. Thus, the acoustic signature can be indicative of the location of the leak.

The acoustic signature may also be indicative of the flow rate of the leak. For example, the amplitude of the acoustic signature of a leak with a relatively higher flow rate will generally be greater than the amplitude of the acoustic signature of a leak with a relatively lower flow rate. Thus, the acoustic signature can be indicative of the flow rate of the leak. In some cases, the flow rate of the leak (which may be determined from the acoustic signature) may itself by indicative of the location of the leak. For example, leaks at a given location within the respiratory therapy system may generally result in higher flow rates than leaks at other locations within the respiratory therapy system. Thus, determining or estimating the flow rate of the leak can in turn aid in determining or estimating the location of the leak within the respiratory therapy system.

In some implementations, data from one or more sensors can be used to determine the location of the leak within the respiratory therapy system. For example, the system may include a plurality of microphones (such as microphone 220 of system 10) positioned at different locations relative to the various components of the respiratory therapy system. Acoustic data generated by the microphones can be used to estimate the location of the leak. For example, the acoustic data can be analyzed to estimate the location of the leak via triangulation, time difference of arrival, steered-response power phase transform, or any other suitable technique or algorithm. Other sensors may also be used, such as particle velocity probes. Additional details related to estimating the location and/or flow rate of the leak can be found in PCT App. No. PCT/IB2022/053332 and PCT/IB2022/050742, each of which is hereby incorporated by reference herein.

In some implementations, method 700 can further include taking an action based on the characterization of the leak. For example, action could be taken in response to the detection of a leak, to the detection of a leak at a certain location within the respiratory therapy system, to the detection of a leak of a certain flow rate, etc. The action can include notifying the user of the leak, such as via an audio message played via a microphone (such as the microphone 220 of the system 10), and/or a visual message displayed on a display device (such as the display device 150 of the respiratory therapy system 100, and/or display device 262 of the user device 260, etc.).

In one example, if it is determined that a leak is originating from the user interface, the action can include notifying the user of the leak, and instructing and/or recommending that the user modify the user interface in some manner. The modification of the user interface can include modifying the position of the user interface on the user's head, modifying the tightness of the user interface, replacing the user interface (with the same type and/or model of user interface, or a different type and/or model of user interface), or other modifications. The modification of the position of the user interface on the user's head can include a modification of the position of the user interface relative to the user's head and/or face, and/or a modification of the position of the user interface relative to absolute space, or some other type of modification of the position of the user interface.

In another example, if it is determined that the leak is originating from the conduit, the action can include notifying the user of the leak, and instructing and/or recommending that the user modify the conduit in some manner. The modification of the conduit can include adjusting a position of the conduit, replacing the conduit (with the same type and/or model of conduit or a different type and/or model of conduit), or other modifications. In a further example, if it is determined that the leak is occurring at the junction of two components of the respiratory therapy system (such as the user interface and the conduit, or the conduit and the respiratory therapy device), the action can include notifying the user of the leak, and instructing and/or recommending that the user adjust/fix the connection between the two components. Any of these examples could be carried out, for example, when a fit test of the user interface is being conducted (e.g., prior to the user's first use of the user interface, prior to the user falling asleep during the sleep session, etc.).

In any of these examples, once the user has modified the user interface, modified the conduit, adjusted the connection between two components of the respiratory therapy device, and/or taken any other instructed and/or recommended action, the respiratory therapy system can be checked to determine if the leak is still occurring. In these examples, method 700 can thus include receiving updated flow data and updated pressure data, comparing the updated flow data and updated pressure data to the predefined pressure versus flow curve (e.g., plotting pressure versus flow data points from the updated flow data and updated pressure data against the predetermined pressure versus flow curve, as shown in FIGS. 8A and 8B), and determining whether the leak has been reduced. Reducing the leak can include eliminating the leak entirely or reducing the flow rate of the leak relative to the flow rate of the leak prior to the modification. Further actions can then be taken, such as providing the user with further modification instructions of the leak is not reduced or not eliminated entirely, providing the user with tips to prevent the reoccurrence of the leak during the sleep session or during subsequent sleep sessions, etc.

In some implementations, the method 700 can further include determining an association between the leak and some event within the sleep session and/or some characteristic of the user and/or the sleep session. For example, additional data from the sleep session can be generated and/or received that is associated with movement of the user during the sleep session, the user's body position during the sleep session, the sleep stage of the user within the sleep session, the time within the sleep session, and other qualities. This additional data, as well as the pressure data and the flow data, can be timestamped, so that it can be determined if there is any type of association between a leak and some event during the sleep session and/or characteristic of the sleep session.

For example, based on the data, it can be determined that a movement event is associated with a leak originating from the respiratory therapy system. The movement event can include the user undergoing a body movement during the sleep session (e.g., the user rolling over, the user moving their arms and/or legs, the user moving their head, etc.). The movement event could additionally or alternatively include the user moving to a body position and/or being in the body position for a predetermined amount of time following the body movement (e.g., the user lying on their stomach, the user lying on their side, the user lying on their back, etc.).

In response to the movement event occurring, different actions can be taken. For example, the action could include sending a recommendation to the user to avoid a body movement and/or a body position during a subsequent sleep session and/or a subsequent portion of the current sleep session. The action could additionally or alternatively include causing the user to move out of a body position. For example, if it is determined that the leak is associated with the user being in an inclined position, the system can cause the user to be moved out of the inclined position, such as by causing the user's bed to return to a flat position or instructing the user to remove one or more pillows.

In some cases, the method 700 can include instructing the user to move out of a body position and/or to undergo a body movement. After the user has followed the instructions, updated pressure and flow data can be received and compared to the predetermined pressure versus flow curve to determine if any reduction in the leak (e.g., a reduced flow rate of the leak or an elimination of the leak). Checking the updated pressure and flow data can accomplish two purposes. First, it can be used to determine if the leak has been mitigated after the user followed the transmitted instructions, so that the system can determine if updated instructions are needed to mitigate the leak now and/or during a subsequent sleep session. Second, it can be used to provide additional information about the leak. For example, whether a certain body movement or body position reduced the leak can aid in determining the location of the leak (e.g., within the user interface and/or conduit vs. within the respiratory therapy device).

The leak can also be associated with certain characteristics of the user and/or of the sleep session. For example, method 700 can include determining (in real-time, delayed during the sleep session, and/or after the sleep session) the various the sleep stages that the user was in during the sleep session. Because the pressure and flow data is time-stamped, the method 700 can include determining what sleep stage the user was in when the leak occurred, and whether there is any correlation between the user's sleep stage and the leak, e.g., whether a leak may occur more frequently when the user is in a REM sleep stage and experiencing REM behavior disorder (RBD). Similarly, the leak can be associated with the time within the sleep session, e.g., whether a leak may occur more frequently later in a sleep session (such as after 5 hours) when the straps of the user interface have become loose. In either implementations, various actions can be taken if a correlation is identified. For example, if it is determined that leaks are occurring during REM sleep stages due to RBD, a more securely fitting user interface can be recommended to the user, or more secure straps/headgear can be recommended for the current user interface. In another example, correlations could be reported to a third party, such as a healthcare provider or a technician. Additional information related to determining the sleep stage of the user can be found in PCT. App. No. PCT/IB2022/054772, which is hereby incorporated by reference herein in its entirety.

In some implementations, comparing the flow data and the pressure data to the predetermined pressure versus flow curve can include additional analysis and/or modification of the flow data and the pressure data. For example, in some implementations, the flow data and the pressure data correspond to periods of time during the sleep session when the user is not breathing, when the user's breathing is not detectable, or when the impact of the user's breathing on the pressure data and flow data is negligible. In some of these implementations, these periods of time occur during the transitions between inspiration and expiration in the user's breathing cycle. Thus, the flow data and the pressure data can be collected at the beginning of inspiration/the end of expiration, and/or the beginning of expiration/the end of inspiration. In others of these implementations, these periods of time occur when the user is intentionally holding their breath. For example, if method 700 is being used to conduct a fit test of the user interface, the user can be instructed to hold their breath for a period of time. While the user is holding their breath, the pressure data and the flow data can be analyzed to determine if a leak is occurring. In any of these implementations, because the flow data and pressure data correspond to times when the user is not breathing or to times when the user's breathing is not detectable or is negligible, any air flow represented by the flow data will exclude flow into and/or out of the user's airway (e.g., via the user's mouth and/or nose). Thus, the flow data and the pressure data can be used to generate pressure versus flow data points, which can be compared to the predetermined pressure versus flow curve.

In other implementations, the flow data and pressure data includes data that is associated with periods of time during the sleep session when the user's breathing is detectable in (e.g., is impacting) the measure flow and pressure values. This breathing-associated data is removed or otherwise discarded, and the remaining flow data and pressure data is then compared to the predetermined pressure versus flow curve. The breathing-associated data can be removed in a variety of ways. For example, in some implementations, the flow data and the pressure data can be averaged over a plurality of breathing cycles (e.g., the average of multiple flow values over a time period is determined, and the average of multiple pressure values over a time period (which may be the same time period as the flow values or a different time period) can be determined), as discussed herein. In some implementations, the time period over which flow values and/or pressure values is averaged is about 30 seconds. Because the average flow rate into and out of the mouth over a plurality of breathing cycles is zero, the remaining flow of air is through any vents in the respiratory therapy system, or through any leaks in the respiratory therapy system. In these implementations, the pressure and flow rate that are compared to the predefined pressure curve can be the average device pressure over the plurality of breathing cycles, and the average flow rate over the plurality of breathing cycles. In other implementations, data associated with the user's breathing can be discarded by using a low-pass filter with a time constant sufficiently long to contain the plurality of breathing cycles, as discussed herein.

In some implementations, the pressure data and flow data can be analyzed directly to estimate the flow rate of the leak. For example, for a given pressure of the motor, the difference between the corresponding flow rate of the predefined pressure versus flow curve (such as the predefined pressure versus flow curve 802 in FIG. 8B) and the corresponding flow rate of a pressure versus flow data point (such as any of the data points 804A-804B in FIG. 8B) can be indicative of the flow rate of the leak.

For example, the flow rate of the leak is given by the equation Qₗₑₐₖ=Q_{d}−Qᵤₛₑᵣ−Qᵥₑₙₜ, where Q_{d} is the device flow rate due to the operation of the motor of the respiratory therapy device, Qᵤₛₑᵣ is the flow rate due to the user's breathing, and Qᵥₑₙₜ is the flow rate through any vents in the respiratory therapy system. Removing the flow rate due to the user's breathing (for example, by averaging flow rate data over multiple breathing cycles) results in Qₗₑₐₖ=Q_{d}−Qᵥₑₙₜ. The vent flow rate Qᵥₑₙₜ can be derived according to Qᵥₑₙₜ=k₁vent \sqrt{Pₘₐₛₖ}+k₂ᵥₑₙₜ·Pₘₐₛₖ, where k₁ᵥₑₙₜ and k₂ᵥₑₙₜ are constants associated with the impedance within the user interface. Pₘₐₛₖ can be derived according to Pₘₐₛₖ=P_{d}−ΔP, where Pa is the device pressure and ΔP is the pressure drop in the respiratory therapy system across the user interface, the conduit, and the humidification tank (if one exists) in the respiratory therapy system. The pressure drop ΔP can be derived according to ΔP=k₁ₛ·Q_{d}²+k₂ₛ·Q_{d}, where k₁ₛ and k₂ₛ are constants associated with the impedance within the user interface, the conduit, and the humidification tank within the respiratory therapy system. Thus, the flow rate of the leak Qₗₑₐₖ can be determined.

Identifying the specific model of the conduit is generally optional. Thus, in some implementations, method 700 does not include step 704, where the specific model of the conduit is identified. In these implementations, after the specific model of the user interface has been identified at step 702, method 700 advances to step 706. At step 706, a predefined pressure versus flow curve that is associated with the identified user interface is selected, and method proceeds with steps 708, 710, and 712. Generally, any of the above features of method 700 can still be used with these implementations.

As the certainty of the identification of the user interface and/or conduit increases, so does the fidelity of the leak characterization. For example, where the user interface and/or conduit are known, a high fidelity leak estimate can be made (such as very small leaks, and varying leaks, being resolvable). Conversely, where the identification of the user interface and/or the conduit is less certain (e.g., based on the fidelity of techniques used to identify the user interface and/or conduit), then the fidelity of the leak characterization is reduced. Thus, in some implementations, the characterization of the leak in the respiratory therapy system can be assigned a probability weighting, which can in turn be used to estimate the success and/or risk of taking some action following the characterization of the leak (e.g., any type of modification to the user interface, the conduit, the respiratory therapy device, etc. that may be undertaken or recommended).

Generally, method 700 (and/or any of the various implementations of method 700 described herein) can be implemented using a system (such as system 10) having a control system (such as control system 200 of system 10) with one or more processors (such as processor 202 of control system 200), and a memory (such as memory device 204 of system 10) storing machine readable instructions. The control system can be coupled to the memory, and method 700 can be implemented when the machine-readable instructions are executed by at least one of the processors of the control system. Method 700 can also be implemented using a computer program product (such as a non-transitory computer readable medium) comprising instructions that when executed by a computer, cause the computer to carry out the steps of method 700.

### ALTERNATIVE IMPLEMENTATIONS

Alternative Implementation 1. A method for characterizing a leak in a respiratory therapy system, the method comprising: identifying a specific model of a user interface of the respiratory therapy system; based on the identified specific model of the user interface, selecting a first one of a plurality of predefined pressure versus flow curves, the first one of the plurality of predefined pressure versus flow curves being associated with airflow characteristics of the respiratory therapy system when the identified specific model of the user interface is used with the respiratory therapy system; receiving pressure data and flow data associated with air flowing in the respiratory therapy system during use of the respiratory therapy system by a user; comparing the pressure data and the flow data to the first one of the plurality of predefined pressure versus flow curves; and characterizing, based at least in part on the comparing, a leak in the respiratory therapy system that occurred during the use of the respiratory therapy system by the user.

Alternative Implementation 2. The method of Alternative Implementation 1, further comprising identifying a specific model of a conduit coupling the user interface to a respiratory therapy device of the respiratory therapy system.

Alternative Implementation 3. The method of Alternative Implementation 2, wherein the selection of the first one of the plurality of predefined pressure versus flow curves is based on the identified specific model of the user interface and the identified specific model of the conduit.

Alternative Implementation 4. The method of Alternative Implementation 2 or Alternative Implementation 3, wherein the first one of the plurality of predefined pressure versus flow curves is associated with airflow characteristics of the respiratory therapy system when the identified specific model of the user interface is coupled to the identified specific model of the conduit.

Alternative Implementation 5. The method of any one of Alternative Implementations 1 to 4, wherein characterizing the leak includes detecting an origin of the leak, estimating a location of the origin of the leak within the respiratory therapy system, determining a flow rate of the leak, or any combination thereof.

Alternative Implementation 6. The method of Alternative Implementation 5, wherein the location of the origin of the leak is within the user interface, at a junction between the user interface and the conduit, within the conduit, at a junction between the conduit and the respiratory therapy device, or within the respiratory therapy device.

Alternative Implementation 7. The method of Alternative Implementation 5 or Alternative Implementation 6, further comprising taking an action based at least in part on the determined location of the origin of the leak, the determined flow rate of the leak, or any combination thereof.

Alternative Implementation 8. The method of any one of Alternative Implementations 5 to 7, further comprising taking an action in response to detecting the leak.

Alternative Implementation 9. The method of Alternative Implementation 8, wherein the action includes transmitting instructions to a user of the respiratory therapy system to modify the user interface, the conduit, or both.

Alternative Implementation 10. The method of Alternative Implementation 9, wherein the instructions to modify the user interface include instructions to replace the user interface, instructions to modify a position of the user interface, instructions to modify a tightness of the user interface, instructions to replace the conduit, instructions to modify a position of the conduit, or any combination thereof.

Alternative Implementation 11. The method of Alternative Implementation 9 or Alternative Implementation 10, further comprising: subsequent to the user modifying the user interface, the conduit, or both, receiving updated pressure data and updated flow data associated with the air flowing in the respiratory therapy system; comparing the updated pressure data and updated flow data to the first one of the plurality of predetermined pressure versus flow curves; and based at least in part on the comparison, determining whether a reduction in the leak occurred.

Alternative Implementation 12. The method of Alternative Implementation 11, wherein the reduction in the leak includes a reduced flow rate of the leak relative to the flow rate of the leak prior to the modification, or an elimination of the leak.

Alternative Implementation 13. The method of any one of Alternative Implementations 4 to 12, wherein the action includes displaying a message on a display device of the respiratory therapy system.

Alternative Implementation 14. The method of any one of Alternative Implementations 1 to 13, wherein a flow rate of the leak is between about 0.001 liters per minute and about 1.0 liters per minute.

Alternative Implementation 15. The method of any one of Alternative Implementations 1 to 14, wherein a flow rate of the leak is less than or equal to about 1.0 liters per minute, or less than or equal to about 5.0 liters per minute.

Alternative Implementation 16. The method of any one of Alternative Implementations 1 to 15, wherein identifying the specific model of the user interface or the specific model of the conduit includes reading a BLE tag positioned within the user interface or coupled to the user interface, reading a BLE tag positioned within the conduit or coupled to the conduit, reading an RFID tag positioned within the user interface or coupled to the user interface, reading an RFID tag positioned within the conduit or coupled to the conduit, scanning a barcode on the user interface, scanning a barcode on the conduit, scanning a QR code on the user interface, scanning a QR code on the conduit, analyzing an image of the user interface to determine the specific model of the user interface, analyzing an image of the conduit to determine the specific model of the conduit, analyzing the pressure data and the flow data to determine the specific model of the user interface, analyzing the pressure data and the flow data to determine the specific model of the conduit, analyzing acoustic data to determine the specific model of the user interface, analyzing acoustic data to determine the specific model of the conduit, or any combination thereof.

Alternative Implementation 17. The method of any one of Alternative Implementations 1 to 16, wherein identifying the specific model of the user interface or the specific model of the conduit includes: generating acoustic data associated with an acoustic reflection of an acoustic signal, the acoustic reflection being indicative of, at least in part, one or more features of the user interface, one or more features of the conduit, or both; analyzing the generated acoustic data; and identifying the specific model of the user interface, the specific model of the conduit, or both, based at least in part, on the analyzed acoustic data.

Alternative Implementation 18. The method of any one of Alternative Implementations 1 to 17, wherein the plurality of predefined pressure versus flow curves included at least 3 pressure versus flow curves, at least 10 pressure versus flow curves, at least 25 pressure versus flow curves, at least 50 pressure versus flow curves, at least 100 pressure versus flow curves, or at least 500 pressure versus flow curves.

Alternative Implementation 19. The method of any one of Alternative Implementations 1 to 18, wherein the pressure data and the flow data correspond to one or more periods of time associated with a transition between an end of inspiration and a beginning of expiration in a breathing cycle of the user, a transition between an end of expiration and a beginning of inspiration in the breathing cycle of the user, or both.

Alternative Implementation 20. The method of Alternative Implementation 19, wherein the comparing includes: determining one or more pressure versus flow rate data points from the pressure data and the flow data; and comparing the one or more pressure versus flow rate data points to the predetermined pressure versus flow curve.

Alternative Implementation 21. The method of any one of Alternative Implementations 1 to 20, wherein the comparing includes: averaging the flow data and the pressure data to determine one or more average flow rate data points and one or more average pressure data points; determining one or more average pressure versus average flow rate data points from the one or more average pressure data points and the one or more average flow rate data points; and comparing the one or more pressure versus flow data points to the predetermined pressure versus flow curve.

Alternative Implementation 22. The method of any one of Alternative Implementations 1 to 21, wherein the comparing includes: discarding a portion of the flow data and the pressure data; determining one or more pressure versus flow rate data points from a remaining portion of the pressure data and the flow data; and comparing the one or more pressure versus flow data points to the predetermined pressure versus flow curve.

Alternative Implementation 23. The method of Alternative Implementation 22, wherein the discarding includes applying a low-pass filter to the pressure data and the flow data.

Alternative Implementation 24. The method of any one of Alternative Implementations 1 to 23, further comprising: determining an association between the leak in the respiratory therapy system and an occurrence of a movement event during the sleep session user; and causing an action to occur based at least in part on the movement event.

Alternative Implementation 25. The method of Alternative Implementation 24, wherein the movement event includes the user undergoing a body movement during the sleep session, the user moving to a different body position during the sleep session, the user remaining in a body position for a predetermined amount of time following a body movement to the body position during the sleep session, or any combination thereof.

Alternative Implementation 26. The method of Alternative Implementation 25, wherein the action includes transmitting, to the user, to a third party, or both, a recommendation to avoid the body movement during a subsequent sleep session or during a subsequent portion of the sleep session.

Alternative Implementation 27. The method of Alternative Implementation 25 or Alternative Implementation 26, wherein the action includes transmitting, to the user, to a third party, or both, a recommendation to avoid the body position during a subsequent sleep session or during a subsequent portion of the sleep session.

Alternative Implementation 28. The method of Alternative Implementation 27, wherein the third party includes a healthcare provider of the user, a technician associated with the respiratory therapy system, or both.

Alternative Implementation 29. The method of any one of Alternative Implementations 25 to 28, wherein the action includes causing the user to move out of the body position during the sleep session.

Alternative Implementation 30. The method of Alternative Implementation 25, further comprising: subsequent to the user moving out of the body position, receiving updated pressure data and updated flow data associated with the air flowing in the respiratory therapy system; comparing the updated pressure data and updated flow data to the first one of the plurality of predetermined pressure versus flow curves; and based at least in part on the comparison, determining whether a reduction in the leak occurred.

Alternative Implementation 31. The method of Alternative Implementation 30, wherein the reduction in the leak includes a reduced flow rate of the leak relative to the flow rate of the leak prior to the user moving out of the body position, or an elimination of the leak.

Alternative Implementation 32. The method of any one of Alternative Implementations 1 to 31, further comprising: determining an association between the leak in the respiratory therapy system and one or more sleep stages of the user during a sleep session when the user is using the respiratory therapy system; and causing an action to occur based at least in part on the one or more sleep stages.

Alternative Implementation 33. The method of any one of Alternative Implementations 1 to 32, further comprising: determining an association between the leak in the respiratory therapy system and a time within a sleep session when the user is using the respiratory therapy system; and causing an action to occur based at least in part on the time within the sleep session.

Alternative Implementation 34. A system for characterizing a leak in a respiratory therapy system, the system comprising: a control system including one or more processors; and a memory having stored thereon machine-readable instructions; wherein the control system is coupled to the memory, and the method of any one of Alternative Implementations 1 to 33 is implemented when the machine-readable instructions in the memory are executed by at least one of the one or more processors of the control system.

Alternative Implementation 35. A system for characterizing a leak in a respiratory therapy system, the system including a control system having one or more processors configured to implement the method of any one of Alternative Implementations 1 to 33.

Alternative Implementation 36. A computer program product comprising instructions which, when executed by a computer, cause the computer to carry out the method of any one of Alternative Implementations 1 to 33.

Alternative Implementation 37. The computer program product of Alternative Implementation 36, wherein the computer program product is a non-transitory computer readable medium.

Alternative Implementation 38. A system for characterizing a leak in a respiratory therapy system, the system comprising: an electronic interface configured to receive data associated with a sleep session of the individual; a memory storing machine-readable instructions; and a control system including one or more processors configured to execute the machine-readable instructions to: identify a specific model of a user interface of the respiratory therapy system; based on the identified specific model of the user interface, select a first one of a plurality of predefined pressure versus flow curves, the first one of the plurality of predefined pressure versus flow curves being associated with airflow characteristics of the respiratory therapy system when the identified specific model of the user interface is used with the respiratory therapy system; receive pressure data and flow data associated with air flowing in the respiratory therapy system during use of the respiratory therapy system by a user; compare the pressure data and the flow data to the first one of the plurality of predefined pressure versus flow curves; and characterize, based at least in part on the comparing, a leak in the respiratory therapy system that occurred during the use of the respiratory therapy system by the user.

Alternative Implementation 39. The system of Alternative Implementation 38, wherein the one or more processors are further configured to execute the machine-readable instructions to identifying a specific model of a conduit coupling the user interface to a respiratory therapy device of the respiratory therapy system.

Alternative Implementation 40. The system of Alternative Implementation 39, wherein the selection of the first one of the plurality of predefined pressure versus flow curves is based on the identified specific model of the user interface and the identified specific model of the conduit.

Alternative Implementation 41. The system of Alternative Implementation 39 or Alternative Implementation 40, wherein the first one of the plurality of predefined pressure versus flow curves is associated with airflow characteristics of the respiratory therapy system when the identified specific model of the user interface is coupled to the identified specific model of the conduit.

Alternative Implementation 42. The system of any one of Alternative Implementations 38 to 41, wherein characterizing the leak includes detecting an origin of the leak, estimating a location of the origin of the leak within the respiratory therapy system, determining a flow rate of the leak, or any combination thereof.

Alternative Implementation 43. The system of Alternative Implementation 42, wherein the location of the origin of the leak is within the user interface, at a junction between the user interface and the conduit, within the conduit, at a junction between the conduit and the respiratory therapy device, or within the respiratory therapy device.

Alternative Implementation 44. The system of Alternative Implementation 42 or Alternative Implementation 43, wherein the one or more processors are further configured to execute the machine-readable instructions to take an action based at least in part on the determined location of the origin of the leak, the determined flow rate of the leak, or any combination thereof.

Alternative Implementation 45. The system of any one of Alternative Implementations 42 to 44, wherein the one or more processors are further configured to execute the machine-readable instructions to take an action in response to detecting the leak.

Alternative Implementation 46. The system of Alternative Implementation 45, wherein the action includes transmitting instructions to a user of the respiratory therapy system to modify the user interface, the conduit, or both.

Alternative Implementation 47. The system of Alternative Implementation 46, wherein the instructions to modify the user interface include instructions to replace the user interface, instructions to modify a position of the user interface, instructions to modify a tightness of the user interface, instructions to replace the conduit, instructions to modify a position of the conduit, or any combination thereof.

Alternative Implementation 48. The system of Alternative Implementation 46 or Alternative Implementation 47, wherein the one or more processors are further configured to execute the machine-readable instructions to: subsequent to the user modifying the user interface, the conduit, or both, receive updated pressure data and updated flow data associated with the air flowing in the respiratory therapy system; compare the updated pressure data and updated flow data to the first one of the plurality of predetermined pressure versus flow curves; and based at least in part on the comparison, determine whether a reduction in the leak occurred.

Alternative Implementation 49. The system of Alternative Implementation 48, wherein the reduction in the leak includes a reduced flow rate of the leak relative to the flow rate of the leak prior to the modification, or an elimination of the leak.

Alternative Implementation 50. The system of any one of Alternative Implementations 41 to 49, wherein the action includes displaying a message on a display device of the respiratory therapy system.

Alternative Implementation 51. The system of any one of Alternative Implementations 38 to 50, wherein a flow rate of the leak is between about 0.001 liters per minute and about 1.0 liters per minute.

Alternative Implementation 52. The system of any one of Alternative Implementations 38 to 51, wherein a flow rate of the leak is less than or equal to about 1.0 liters per minute, or less than or equal to about 5.0 liters per minute.

Alternative Implementation 53. The system of any one of Alternative Implementations 38 to 52, wherein identifying the specific model of the user interface or the specific model of the conduit includes reading a BLE tag positioned within the user interface or coupled to the user interface, reading a BLE tag positioned within the conduit or coupled to the conduit, reading an RFID tag positioned within the user interface or coupled to the user interface, reading an RFID tag positioned within the conduit or coupled to the conduit, scanning a barcode on the user interface, scanning a barcode on the conduit, scanning a QR code on the user interface, scanning a QR code on the conduit, analyzing an image of the user interface to determine the specific model of the user interface, analyzing an image of the conduit to determine the specific model of the conduit, analyzing the pressure data and the flow data to determine the specific model of the user interface, analyzing the pressure data and the flow data to determine the specific model of the conduit, analyzing acoustic data to determine the specific model of the user interface, analyzing acoustic data to determine the specific model of the conduit, or any combination thereof.

Alternative Implementation 54. The system of any one of Alternative Implementations 38 to 53, wherein identifying the specific model of the user interface or the specific model of the conduit includes: generating acoustic data associated with an acoustic reflection of an acoustic signal, the acoustic reflection being indicative of, at least in part, one or more features of the user interface, one or more features of the conduit, or both; analyzing the generated acoustic data; and identifying the specific model of the user interface, the specific model of the conduit, or both, based at least in part, on the analyzed acoustic data.

Alternative Implementation 55. The system of any one of Alternative Implementations 38 to 54, wherein the plurality of predefined pressure versus flow curves included at least 3 pressure versus flow curves, at least 10 pressure versus flow curves, at least 25 pressure versus flow curves, at least 50 pressure versus flow curves, at least 100 pressure versus flow curves, or at least 500 pressure versus flow curves.

Alternative Implementation 56. The system of any one of Alternative Implementations 38 to 55, wherein the pressure data and the flow data correspond to one or more periods of time associated with a transition between an end of inspiration and a beginning of expiration in a breathing cycle of the user, a transition between an end of expiration and a beginning of inspiration in the breathing cycle of the user, or both.

Alternative Implementation 57. The system of Alternative Implementation 56, wherein the comparing includes: determining one or more pressure versus flow rate data points from the pressure data and the flow data; and comparing the one or more pressure versus flow rate data points to the predetermined pressure versus flow curve.

Alternative Implementation 58. The system of any one of Alternative Implementations 38 to 57, wherein the comparing includes: averaging the flow data and the pressure data to determine one or more average flow rate data points and one or more average pressure data points; determining one or more average pressure versus average flow rate data points from the one or more average pressure data points and the one or more average flow rate data points; and comparing the one or more pressure versus flow data points to the predetermined pressure versus flow curve.

Alternative Implementation 59. The system of any one of Alternative Implementations 38 to 58, wherein the comparing includes: discarding a portion of the flow data and the pressure data; determining one or more pressure versus flow rate data points from a remaining portion of the pressure data and the flow data; and comparing the one or more pressure versus flow data points to the predetermined pressure versus flow curve.

Alternative Implementation 60. The system of Alternative Implementation 59, wherein the discarding includes applying a low-pass filter to the pressure data and the flow data.

Alternative Implementation 61. The system of any one of Alternative Implementations 38 to 60, wherein the one or more processors are further configured to execute the machine-readable instructions to: determine an association between the leak in the respiratory therapy system and an occurrence of a movement event during the sleep session user; and cause an action to occur based at least in part on the movement event.

Alternative Implementation 62. The system of Alternative Implementation 61, wherein the movement event includes the user undergoing a body movement during the sleep session, the user moving to a different body position during the sleep session, the user remaining in a body position for a predetermined amount of time following a body movement to the body position during the sleep session, or any combination thereof.

Alternative Implementation 63. The system of Alternative Implementation 62, wherein the action includes transmitting, to the user, to a third party, or both, a recommendation to avoid the body movement during a subsequent sleep session or during a subsequent portion of the sleep session.

Alternative Implementation 64. The system of Alternative Implementation 62 or Alternative Implementation 63, wherein the action includes transmitting, to the user, to a third party, or both, a recommendation to avoid the body position during a subsequent sleep session or during a subsequent portion of the sleep session.

Alternative Implementation 65. The system of Alternative Implementation 64, wherein the third party includes a healthcare provider of the user, a technician associated with the respiratory therapy system, or both.

Alternative Implementation 66. The system of any one of Alternative Implementations 62 to 65, wherein the action includes causing the user to move out of the body position during the sleep session.

Alternative Implementation 67. The system of Alternative Implementation 62, wherein the one or more processors are further configured to execute the machine-readable instructions to: subsequent to the user moving out of the body position, receive updated pressure data and updated flow data associated with the air flowing in the respiratory therapy system; compare the updated pressure data and updated flow data to the first one of the plurality of predetermined pressure versus flow curves; and based at least in part on the comparison, determine whether a reduction in the leak occurred.

Alternative Implementation 68. The system of Alternative Implementation 67, wherein the reduction in the leak includes a reduced flow rate of the leak relative to the flow rate of the leak prior to the user moving out of the body position, or an elimination of the leak.

Alternative Implementation 69. The system of any one of Alternative Implementations 38 to 68, wherein the one or more processors are further configured to execute the machine-readable instructions to: determine an association between the leak in the respiratory therapy system and one or more sleep stages of the user during a sleep session when the user is using the respiratory therapy system; and cause an action to occur based at least in part on the one or more sleep stages.

Alternative Implementation 70. The system of any one of Alternative Implementations 38 to 69, wherein the one or more processors are further configured to execute the machine-readable instructions to: determine an association between the leak in the respiratory therapy system and a time within a sleep session when the user is using the respiratory therapy system; and cause an action to occur based at least in part on the time within the sleep session.

One or more elements or aspects or steps, or any portion(s) thereof, from one or more of any of the Alternative Implementations and/or claims herein can be combined with one or more elements or aspects or steps, or any portion(s) thereof, from one or more of any of the other Alternative Implementations and/or claims herein or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for characterizing a leak in a respiratory therapy system, the method comprising:
identifying a specific model of a user interface of the respiratory therapy system;
based on the identified specific model of the user interface, selecting a first one of a plurality of predefined pressure versus flow curves, the first one of the plurality of predefined pressure versus flow curves being associated with airflow characteristics of the respiratory therapy system when the identified specific model of the user interface is used with the respiratory therapy system;
receiving pressure data and flow data associated with air flowing in the respiratory therapy system during use of the respiratory therapy system by a user;
comparing the pressure data and the flow data to the first one of the plurality of predefined pressure versus flow curves; and
characterizing, based at least in part on the comparing, a leak in the respiratory therapy system that occurred during the use of the respiratory therapy system by the user.

2-4. (canceled)

5. The method of claim 1, wherein characterizing the leak includes detecting an origin of the leak, estimating a location of the origin of the leak within the respiratory therapy system, determining a flow rate of the leak, or any combination thereof, and wherein the method further comprises taking an action in response to detecting the leak, based at least in part on the estimated location of the origin of the leak, the determined flow rate of the leak, or any combination thereof, wherein the action includes at least (i) transmitting instructions to the user of the respiratory therapy system to modify the user interface, a conduit coupling the user interface to a respiratory therapy device of the respiratory therapy system, or both, (ii) causing a message to be displayed on a display device of the respiratory therapy system, (iii) both (i) and (ii).

6-13. (canceled)

14. The method of claim 1, wherein a flow rate of the leak is (i) between about 0.001 liters per minute and about 1.0 liters per minute, (ii) less than or equal to about 1.0 liters per minute, or (iii) less than or equal to about 5.0 liters per minute.

15. (canceled)

16. The method of claim 1, wherein identifying the specific model of the user interface or a specific model of a conduit coupling the user interface to a respiratory therapy device of the respiratory therapy system includes reading a BLE tag positioned within the user interface or coupled to the user interface, reading a BLE tag positioned within the conduit or coupled to the conduit, reading an RFID tag positioned within the user interface or coupled to the user interface, reading an RFID tag positioned within the conduit or coupled to the conduit, scanning a barcode on the user interface, scanning a barcode on the conduit, scanning a QR code on the user interface, scanning a QR code on the conduit, analyzing an image of the user interface to determine the specific model of the user interface, analyzing an image of the conduit to determine the specific model of the conduit, analyzing the pressure data and the flow data to determine the specific model of the user interface, analyzing the pressure data and the flow data to determine the specific model of the conduit, analyzing acoustic data to determine the specific model of the user interface, analyzing acoustic data to determine the specific model of the conduit, or any combination thereof.

17. The method of claim 1, wherein identifying the specific model of the user interface or a specific model of a conduit coupling the user interface to a respiratory therapy device of the respiratory therapy system includes:
generating acoustic data associated with an acoustic reflection of an acoustic signal, the acoustic reflection being indicative of, at least in part, one or more features of the user interface, one or more features of the conduit, or both;
analyzing the generated acoustic data; and
identifying the specific model of the user interface, the specific model of the conduit, or both, based at least in part, on the analyzed acoustic data.

18-23. (canceled)

24. The method of claim 1, further comprising:
determining an association between the leak in the respiratory therapy system and (i) an occurrence of a movement event during a sleep session when the user is using the respiratory therapy system, (ii) one or more sleep stages of the user during the sleep session, (iii) a time within the sleep session, or (iv) any combination of (i)-(iii); and
causing an action to occur based at least in part on the movement event.

25-37. (canceled)

38. A system for characterizing a leak in a respiratory therapy system, the system comprising:
an electronic interface configured to receive pressure data and flow data associated with air flowing in the respiratory therapy system during use of the respiratory therapy system by a user;
a memory storing machine-readable instructions; and
a control system including one or more processors configured to execute the machine-readable instructions to:
identify a specific model of a user interface of the respiratory therapy system;
based on the identified specific model of the user interface, select a first one of a plurality of predefined pressure versus flow curves, the first one of the plurality of predefined pressure versus flow curves being associated with airflow characteristics of the respiratory therapy system when the identified specific model of the user interface is used with the respiratory therapy system;
receive pressure data and flow data associated with air flowing in the respiratory therapy system during use of the respiratory therapy system by a user;
compare the pressure data and the flow data to the first one of the plurality of predefined pressure versus flow curves; and
characterize, based at least in part on the comparing, a leak in the respiratory therapy system that occurred during the use of the respiratory therapy system by the user.

39. The system of claim 38, wherein the one or more processors are further configured to execute the machine-readable instructions to identifying a specific model of a conduit coupling the user interface to a respiratory therapy device of the respiratory therapy system, and wherein the selection of the first one of the plurality of predefined pressure versus flow curves is based on the identified specific model of the user interface and the identified specific model of the conduit.

40. (canceled)

41. The system of claim 39, wherein the first one of the plurality of predefined pressure versus flow curves is associated with airflow characteristics of the respiratory therapy system when the identified specific model of the user interface is coupled to the identified specific model of the conduit.

42. The system of claim 38, wherein characterizing the leak includes detecting an origin of the leak, estimating a location of the origin of the leak within the respiratory therapy system, determining a flow rate of the leak, or any combination thereof.

43. (canceled)

44. The system of claim 42, wherein the one or more processors are further configured to execute the machine-readable instructions to take an action in response to detecting the leak, based at least in part on the estimated location of the origin of the leak, the determined flow rate of the leak, or any combination thereof, wherein the action includes at least (i) transmitting instructions to the user of the respiratory therapy system to modify the user interface, a conduit coupling the user interface to a respiratory therapy device of the respiratory therapy system, or both, (ii) causing a message to be displayed on a display device of the respiratory therapy system, (iii) both (i) and (ii).

45-46. (canceled)

47. The system of claim 44, wherein the instructions to modify the user interface include instructions to replace the user interface, instructions to modify a position of the user interface, instructions to modify a tightness of the user interface, instructions to replace the conduit, instructions to modify a position of the conduit, or any combination thereof.

48. The system of claim 47, wherein the one or more processors are further configured to execute the machine-readable instructions to:
subsequent to the user modifying the user interface, the conduit, or both, receive updated pressure data and updated flow data associated with the air flowing in the respiratory therapy system;
compare the updated pressure data and updated flow data to the first one of the plurality of predefined pressure versus flow curves; and
based at least in part on the comparison, determine whether a reduction in the leak occurred, the reduction in the leak including a reduced flow rate of the leak relative to the flow rate of the leak prior to the modification, or an elimination of the leak.

49-50. (canceled)

51. The system of claim 38, wherein a flow rate of the leak is (i) between about 0.001 liters per minute and about 1.0 liters per minute, (ii) less than or equal to about 1.0 liters per minute, or (iii) less than or equal to about 5.0 liters per minute.

52. (canceled)

53. The system of claim 38, wherein identifying the specific model of the user interface or a specific model of a conduit coupling the user interface to a respiratory therapy device of the respiratory therapy system includes reading a BLE tag positioned within the user interface or coupled to the user interface, reading a BLE tag positioned within the conduit or coupled to the conduit, reading an RFID tag positioned within the user interface or coupled to the user interface, reading an RFID tag positioned within the conduit or coupled to the conduit, scanning a barcode on the user interface, scanning a barcode on the conduit, scanning a QR code on the user interface, scanning a QR code on the conduit, analyzing an image of the user interface to determine the specific model of the user interface, analyzing an image of the conduit to determine the specific model of the conduit, analyzing the pressure data and the flow data to determine the specific model of the user interface, analyzing the pressure data and the flow data to determine the specific model of the conduit, analyzing acoustic data to determine the specific model of the user interface, analyzing acoustic data to determine the specific model of the conduit, or any combination thereof.

54. The system of claim 38, wherein identifying the specific model of the user interface or a specific model of a conduit coupling the user interface to a respiratory therapy device of the respiratory therapy system includes:
generating acoustic data associated with an acoustic reflection of an acoustic signal, the acoustic reflection being indicative of, at least in part, one or more features of the user interface, one or more features of the conduit, or both;
analyzing the generated acoustic data; and
identifying the specific model of the user interface, the specific model of the conduit, or both, based at least in part, on the analyzed acoustic data.

55. The system of claim 38, wherein the plurality of predefined pressure versus flow curves included at least 3 pressure versus flow curves, at least 10 pressure versus flow curves, at least 25 pressure versus flow curves, at least 50 pressure versus flow curves, at least 100 pressure versus flow curves, or at least 500 pressure versus flow curves.

56-57. (canceled)

58. The system of claim 38, wherein the comparing includes:
averaging the flow data and the pressure data to determine one or more average flow rate data points and one or more average pressure data points;
determining one or more average pressure versus average flow rate data points from the one or more average pressure data points and the one or more average flow rate data points; and
comparing the one or more pressure versus flow data points to the first one of the plurality of predefined pressure versus flow curve.

59-60. (canceled)

61. The system of claim 38, wherein the one or more processors are further configured to execute the machine-readable instructions to:
determine an association between the leak in the respiratory therapy system and an occurrence of a movement event during a sleep session when the user is using the respiratory therapy system; and
cause an action to occur based at least in part on the movement event.

62. The system of claim 61, wherein the movement event includes the user undergoing a body movement during the sleep session, the user moving to a different body position during the sleep session, the user remaining in a body position for a predetermined amount of time following a body movement to the body position during the sleep session, or any combination thereof.

63. The system of claim 62, wherein the action includes (i) transmitting, to the user, to a third party, or both, a recommendation to avoid the body movement during a subsequent sleep session or during a subsequent portion of the sleep session, (ii) transmitting, to the user, to the third party, or both, a recommendation to avoid the body position during the subsequent sleep session or during the subsequent portion of the sleep session, (iii) causing the user to move out of the body position during the sleep session.

64-66. (canceled)

67. The system of claim 62, wherein the one or more processors are further configured to execute the machine-readable instructions to:
subsequent to the user moving out of the body position, receive updated pressure data and updated flow data associated with the air flowing in the respiratory therapy system;
compare the updated pressure data and updated flow data to the first one of the plurality of predefined pressure versus flow curves; and
based at least in part on the comparison, determine whether a reduction in the leak occurred, the reduction in the leak including a reduced flow rate of the leak relative to a flow rate of the leak prior to the user moving out of the body position, or an elimination of the leak.

68. (canceled)

69. The system of claim 38, wherein the one or more processors are further configured to execute the machine-readable instructions to:
determine an association between the leak in the respiratory therapy system and (i) one or more sleep stages of the user during a sleep session when the user is using the respiratory therapy system, (ii) a time within the sleep session when the user is using the respiratory therapy system, or (iii) both (i) and (ii); and
cause an action to occur based at least in part on the one or more sleep stages.

70. (canceled)
